(19) United States
(12) Patent Application Publication
(10) Pub. No.: US 2023/0043363 A1
(43) Pub. Date: Feb. 9, 2023

(54) ARTIFICIAL INTELLIGENCE BASED MATERIAL SCREENING FOR TARGET PROPERTIES

(71) Applicant: International Business Machines Corporation, Armonk, NY (US)

(72) Inventors: Rodrigo Neumann Barros Ferreira, Rio de Janeiro (BR); Fausto Martelli, Stockton Heath (GB); BREANNDAN O'CONCHUIR, Warrington (GB); Tonia Elengikal, Long Island City, NY (US); Binquan Luan, Chappaqua, NY (US); Ronaldo Giro, Americana (BR); Mathias B. Steiner, Rio de Janeiro (BR); Anshul Gupta, Valhalla, NY (US)

(21) Appl. No.: 17/397,046

(22) Filed: Aug. 9, 2021

(51) Int. Cl. G16C 20/70; G16C 20/30; G16C 60/00; G06F 30/27

(52) U.S. Cl. CPC G16C 20/70; G16C 20/30; G16C 60/00; G06F 30/27; G01N 23/2055

(57) ABSTRACT

A material screening process of generating input features for each material of a subset of materials to be screened, generating target properties for each material of the subset of materials, inputting screening conditions, the input features, and the target properties into a material screening artificial intelligence model and training the material screening artificial intelligence model based on the inputs. Once the model is trained, inputting a dataset of materials to be screened into the trained material screening artificial intelligence model, the dataset of materials includes the subset of materials used to train the model, screening the dataset of materials on the trained material screening artificial intelligence model using the screening conditions and ranking the materials of the dataset based on predicted target properties obtained from the screening.

### BACKGROUND

This disclosure is directed to computers, and computer applications, and more particularly to computer-implemented methods and systems for material screening for target properties.

The discovery of optimized materials for carbon capture requires analyzing CO₂ adsorption of nano-porous materials at a range of temperature and pressure conditions. This task is computationally intensive, and it is impractical to perform physics-based simulations for the millions of materials candidates. A drawback of known solutions is that existing materials screening approaches contain a top layer in which rapid geometric and topological characterizations of the materials are deployed only to eliminate samples having less favorable adsorption properties. By using this approach, only the properties of the most promising material candidates are subsequently calculated using molecular dynamics simulation, significantly reducing discovery time and computational cost. However, the top layer topological and geometric descriptors can only classify samples for elimination or further study. The existing screening methods neglect the intricate chemical interactions between various atomic species present in the nanopore framework and gas phase, thus limiting the effectiveness of such descriptors as a screening tool.

### SUMMARY

One embodiment of a computer implemented method for material screening includes the steps of generating input features for each material of a subset of materials to be screened, generating target properties for each material of the subset of materials, inputting screening conditions, the input features, and the target properties into a material screening artificial intelligence model and training the material screening artificial intelligence model based on the inputs. Once the model is trained, inputting a dataset of materials to be screened into the trained material screening artificial intelligence model, the dataset of materials may include the subset of materials used to train the model, screening the dataset of materials on the trained material screening artificial intelligence model using the screening conditions and ranking the materials of the dataset based on predicted target properties obtained from the screening. In some embodiments, the method includes training a neurosymbolic material screening model using predicted target properties, neurosymbolic axioms and the screening conditions, extracting analytical expressions of the target properties from the trained neurosymbolic material screening model, evaluating the extracted analytical expressions using a process efficiency model and calculating a process efficiency score.

A system that includes one or more processors operable to perform one or more methods described herein also may be provided.

A computer readable storage medium storing a program of instructions executable by a machine to perform one or more methods described herein also may be provided.

Further features as well as the structure and operation of various embodiments are described in detail below with reference to the accompanying drawings. In the drawings, like reference numbers indicate identical or functionally similar elements.

### BRIEF DESCRIPTION OF THE DRAWINGS

FIG. 1 is a flow diagram of one embodiment of the methods disclosed in this specification.

FIG. 2 is a flow diagram of one embodiment of the methods disclosed in this specification.

FIG. 3 is a flow diagram of one embodiment of the method for compiling the material subset.

FIG. 4 is a flow diagram of one embodiment of the method for calculation/simulation of adsorption, geometric and topological metrics.

FIG. 5 is a flow diagram of one embodiment of the method for training of an AI machine-learning model.

FIG. 6 is a flow diagram of one embodiment of the method for materials screening that includes training a neurosymbolic AI model.

FIG. 7A is a flow diagram of one embodiment of the method for generating adsorption training data for training a neurosymbolic AI model.

FIG. 7B is a flow diagram of one embodiment of the method for neurosymbolic AI training.

FIG. 7C is a flow diagram of one embodiment of the method for process optimization of material rankings using a neurosymbolic AI model.

FIG. 7D is a flow diagram of one embodiment of the method for process optimization of flue gas composition using a neurosymbolic AI model.

FIG. 7E is a flow diagram of one embodiment of the method for process optimization of pressure/temperature ranges using a neurosymbolic AI model.

FIG. 8 is a block diagram of one embodiment of a cloud infrastructure for implementing the materials screening methods disclosed herein.

FIG. 9 depicts a cloud computing environment according to an embodiment of the present invention.

FIG. 10 depicts abstraction model layers according to an embodiment of the present invention.

FIG. 11 is a block diagram of an exemplary computing system suitable for implementation of the embodiments of the invention disclosed in this specification.

### DETAILED DESCRIPTION

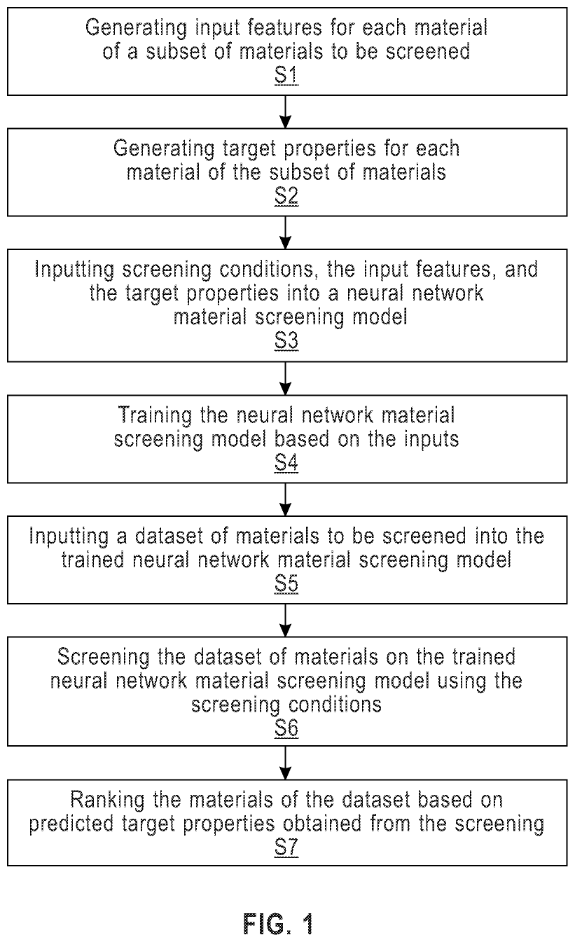

FIG. 1 is a flow diagram of one embodiment of a material screening process. The process includes step S1 of generating input features for each material of a subset of materials to be screened and step S2 of generating target properties for each material of the subset of materials. Next, the process includes step S3 of inputting screening conditions, the input features, and the target properties into a material screening artificial intelligence model and step S4 of training the material screening artificial intelligence model based on the inputs. Once the model is trained, the process includes step S5 of inputting a dataset of materials to be screened into the trained material screening artificial intelligence model. The dataset of materials includes the subset of materials used to train the model. Accordingly, the dataset of materials is larger than the subset of materials. The process flow then includes step S6 of screening the dataset of materials on the trained material screening artificial intelligence model using the screening conditions and then step S7 of ranking the materials of the dataset based on predicted target properties obtained from the screening.

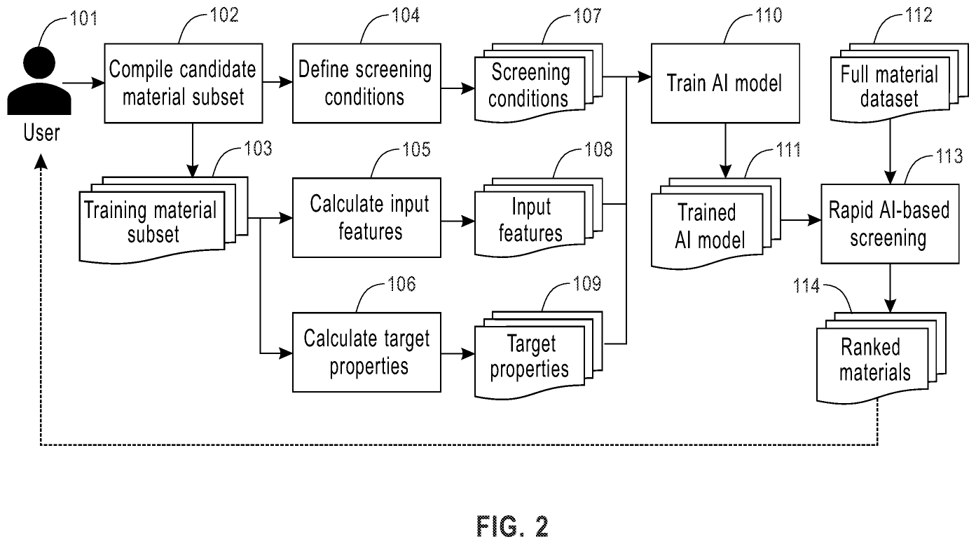

As shown in more detail in the flow diagram of FIG. 2, in one embodiment of the material screening process, a user 101 compiles a subset of candidate materials 102 for creating a training subset of material definitions 103. The training subset of candidate material 103 may be a small set of materials that are part of a much larger set of materials to be screened. The user then defines the screening conditions 104 that might include pressures, temperatures and flue gas compositions 107. The training material subset 103 is subjected to a series of calculations 105 and 106 to be described below that extract the input features 108 and target properties 109. The screening conditions 107, input features 108 and target properties 109 are used to train an artificial intelligence (AI) model 110. The trained AI model 111 can then be used as part of a Rapid AI-based screening workflow 113 to process the much larger full material dataset 112 and rank those materials in terms of their target properties 114.

Artificial intelligence (AI) is a class of technology that mimics human intelligence to predict, automate, and optimize tasks that humans have historically done. Machine learning is a subfield of artificial intelligence and deep learning is a subfield of machine learning. Neural networks make up the backbone of the learning algorithms. Neural networks mimic the human brain through a set of algorithms. At a basic level, a neural network is comprised of four main components: inputs, weights, a bias or threshold, and an output. Machine learning is based on computer algorithms that improve automatically through experience and by the use of data. Machine learning algorithms build a model based on sample data, known as training data, in order to make predictions or decisions without being explicitly programmed to do so. Most often, the training processes a large amount of data through the algorithm to maximize likelihood or minimize cost, yielding a trained model. Analyzing data from many wells in different conditions, the model learns to detect all the types of patterns and distinguish these from normal operation. The AI model 110 may be any type of machine learning model, including a neural network or deep learning.

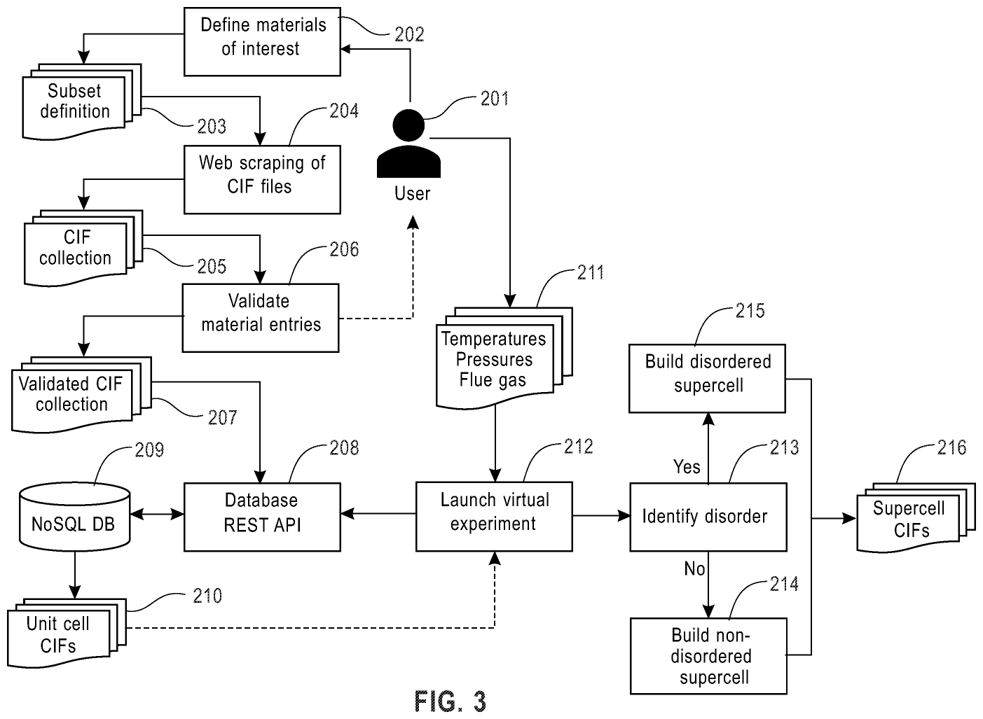

FIG. 3 expands on steps 102, 103, 104 and 107 of FIG. 2 for compiling the material subset. FIG. 3 focuses on the compilation of a collection of crystal structure files that serve as input for the screening method. In some embodiments, the user 201 starts the interaction with the system by defining a subset of materials of interest to be analysed 202. This subset 203 could, for instance, be defined by a certain class of nanoporous materials (i.e., zeolites, metal-organic frameworks, zeolitic imidazolate frameworks, covalent organic framework or porous polymer networks, etc). Once the subset of interest 203 is defined, a collection of web scrappers 204 extract data from known sources of information for that particular class of materials. In some embodiment, the sources can either be proprietary (internal databases) or open-source repositories, with licenses such as creative common copyright licenses. Each material of interest is defined as a Crystallographic Information File (CIF). The result of the web scrappers is to retrieve a collection of CIFs 205 or alternative file formats that are later converted to CIF.

Thereafter, a validation step 206 requires the user to assert the suitability of the CIF collection by taking into consideration the number and completeness of the CIFs. In some embodiments, a complete CIF is one that includes, at least: a) crystal cell lengths and angles; b) symmetry group, symmetry number or list of symmetry operations; c) atom type and fractional coordinates. An invalid CIF may be removed from the collection. If the number of valid CIFs in the collection is deemed insufficient, the collection will need to be enlarged by selecting different materials of interest. In some embodiments, the validated CIF collection 207 is then inserted through a REST API 208 into a NoSQL Database 209 alongside metadata pertaining to their name, class (as in 202) and source (as in 204).

A REST API (also known as RESTful API) is an application programming interface (API or web API) that conforms to the constraints of REST architectural style and allows for interaction with RESTful web services. REST stands for representational state transfer. REST is a set of architectural constraints. When a client request is made via a RESTful API, it transfers a representation of the state of the resource to the requester or endpoint. This information, or representation, is delivered in one of several formats via HTTP: JSON (Javascript Object Notation), HTML, XLT, Python, PHP, or plain text. A NoSQL (also referred to as “non-SQL” or “non-relational”) database provides a mechanism for storage and retrieval of data that is modeled in means other than the tabular relations used in relational databases.

The user 201 provides the external conditions 211 under which the screening must take place: temperature(s), pressure(s) and flue gas composition(s). The system then launches a virtual experiment 212 that will retrieve from the database 209 a set of CIFs 210 representing the unit cell of the adsorbent materials under study. Each CIF is scanned for crystallographic disorder 213. Disorder is encoded in the CIF as fractional occupancies for some atom sites.

In some embodiments, if the material has no disorder, a supercell is built 214 by building a stoichiometrically-balanced supercell with the appropriate size by replicating the unit cell as many times as necessary to avoid self-interactions. In one embodiment, the supercell may be built by replicating the unit cell as many times as needed to ensure that all perpendicular cell lengths are at least twice as large as the cut-off radius for atom-atom interactions—typically, 12-13 Å. Supercell is a software program which has been designed to facilitate the construction of structural models for the description of vacancy or substitution defects in otherwise periodically-ordered (crystalline) materials. The software program includes algorithms for structure manipulation, supercell generation, permutations of atoms and vacancies, charge balancing, detecting symmetry-equivalent structures, Coulomb energy calculations and sampling output configurations. If the material has disorder, the unit cell is further replicated and randomised 215 to ensure stoichiometric balance and the variant with the lowest electrostatic energy is selected. In either case, the result is a collection of CIFs representing the appropriate supercell 216 required for subsequent calculations.

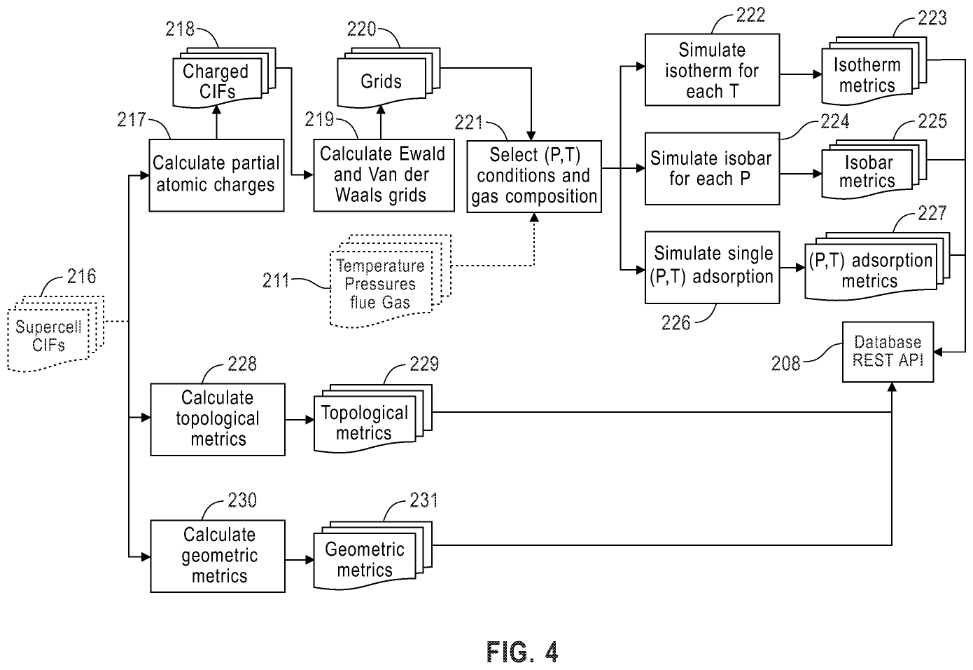

FIG. 4 expands on steps 105, 106, 108 and 109 of FIG. 2. FIG. 4 focuses on the calculation/simulation of adsorption, geometric and topological figures-of-merit which are later stored in a database. In some embodiments, each supercell CIF 216 is processed to compute their adsorption in steps 217-227, topological properties in steps 228-229 and geometric properties in steps 230-231. In one embodiment, the computation of the adsorption properties of each material proceeds first with charges being assigned to each atom in the supercell 217 using methods such as Qeq, EQeq, DDEC, among others. For example, charge equilibration (Qeq) methods can estimate the electrostatic potential of molecules and periodic frameworks by assigning point charges to each atom, using only a small fraction of the resources needed to compute density functional (DFT)-derived charges which may make possible the computational screening of thousands of microporous structures to assess their performance for the adsorption of polar molecules. If the CIF already contains atomic charge information, those can also be used. In either case the result will be a CIF augmented with atomic charge information 218.

Next, in some embodiments, electrostatic Ewald and van der Waals grids are calculated 219 leading to energy grid files 220 that can greatly speed up the subsequent simulation. The pressure, temperature and flue gas composition for the simulation are loaded 221 and a simulation is launched. Depending on the user input 211 the simulation will comprise an adsorption isotherm (222), an adsorption isobar (224) or an adsorption simulation for a single pressure and temperature value 226. In any case, the resulting isotherm 223, isobar 225 or single pressure-temperature (P,T) adsorption metric 227 is stored via the REST API 208 into the NoSQL database 209. An adsorption isotherm curve represents the variation of the amount of gas adsorbed by a material as a function of pressure for a given temperature. There is no limitation on the type or form of isotherm as an isotherm curve may have different shapes, e.g. types I-VI and other shapes and forms not described here, which lead to different functional forms when searching for an analytical expression. An adsorption isobar curve represents the variation of the amount of gas adsorbed by a material as a function of temperature for a given pressure.

The adsorption properties of these materials can be simulated using, in one example, the Grand Canonical Monte Carlo simulation method as implemented by various open-source programs such as Cassandra, DL Monte, and others. Likewise, the topological properties of the material can be calculated from their respective CIFs 228 and the resulting topological metrics 229 stored via the REST API 208 into the NoSQL database 209. These methods may be implemented in open-source packages such as Ripser and Mapper. In Ripser, the shape of the framework structure is encoded within data representations emanating from persistent homology. In the Mapper package, similarity metrics can be introduced to quantify how similar or dissimilar any two high-performing material data representations are, while clustering techniques can be applied to both identify and predict the type of nanoporous structures that have good adsorption properties.

Thereafter, in some embodiments, the geometric properties are calculated from the CIFs 230 and the resulting geometric metrics 231 are stored via the REST API 208 into the NoSQL database 209. These properties can be computed by applying geometry-based analysis of structure and topology of the void space within nanoporous materials. For example, the system can apply algorithms such as Voronoi decomposition, which for a given arrangement of atoms in a periodic domain provides a graph representation of the void space. The resulting Voronoi network can then by examined to extract geometric figures of merit relevant to an incoming spherical probe sphere representing a carbon doxide molecule. Examples include: the crystal porosity/density, accessible surface area, accessible volume, diameter of largest free sphere, diameter of largest included sphere and diameter of largest included sphere along free path. The geometric calculation methods are implemented in open-source packages, such as Zeo++ and PoreBlazer.

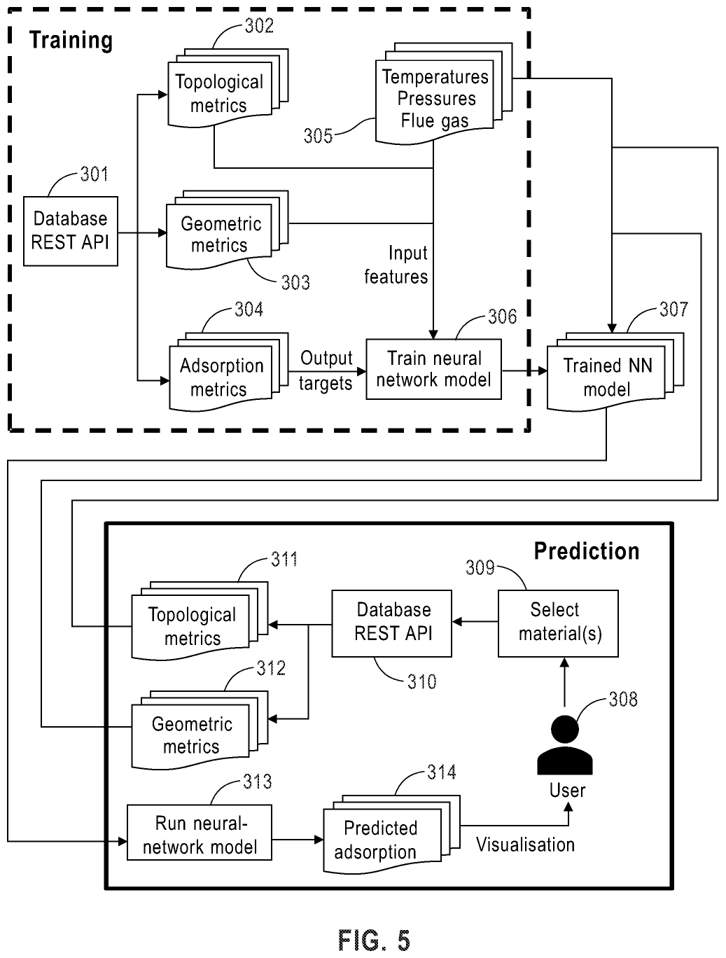

FIG. 5 expands on steps 110, 111, 112, 113 and 114 of FIG. 2. FIG. 5 focuses on the training of an AI machine-learning model and its later application to predict adsorption properties. In some embodiments, a neural network is trained on the set of simulated data produced in FIGS. 3 and 4. Initially, the database REST API 301 is used to extract the previously computed topological parameters 302, geometric parameters 303, and the set of adsorbate gas composition and pressure/temperature conditions 305. These entities fully characterize the input data entered in the neural network. Next, the neural network is trained at 306. In some embodiments, the network configurations and weights are optimized by fitting the predicted adsorption results to the previously computed target results 304 written in the database 301. Once training has been completed, the trained neural network model 307 acts as a surrogate to the computer simulation, operating on the same input to produce the same output.

In the prediction step, the trained neural network 307 can be implemented to perform a rapid screening of potentially millions of materials of interest. User 308 selects materials at 309. The topological metrics 311 and geometric metrics 312 of the selected materials are obtained from the database 310 and are input to the trained neural network model 307 and the model is run at 313 to screen for the target materials. The predicted adsorption data is output at 314 and may be displayed to the user.

In some embodiments, the disclosed methods can be extended by using concepts from neurosymbolic AI in order to learn functional expressions describing how the adsorption properties vary under changing environmental conditions. In one embodiment, canonical expressions for adsorption isobars and isotherms corresponding to the physical process of loading and unloading the captured carbon dioxide molecules from the nanoporous adsorbate material at different pressures and temperatures can be determined. The shape of these isotherms and isobars is intrinsically linked to the efficiency of the process and can be scored/ranked accordingly. In one embodiment, this extension is deployed as a secondary hierarchical screening layer. The methods described above can be used to screen through large databases containing up to millions of candidate nanoporous materials. Then only a certain percentage of these screened materials would then progress to this secondary workflow which assesses the performance of the materials in realistic process engineering environments.

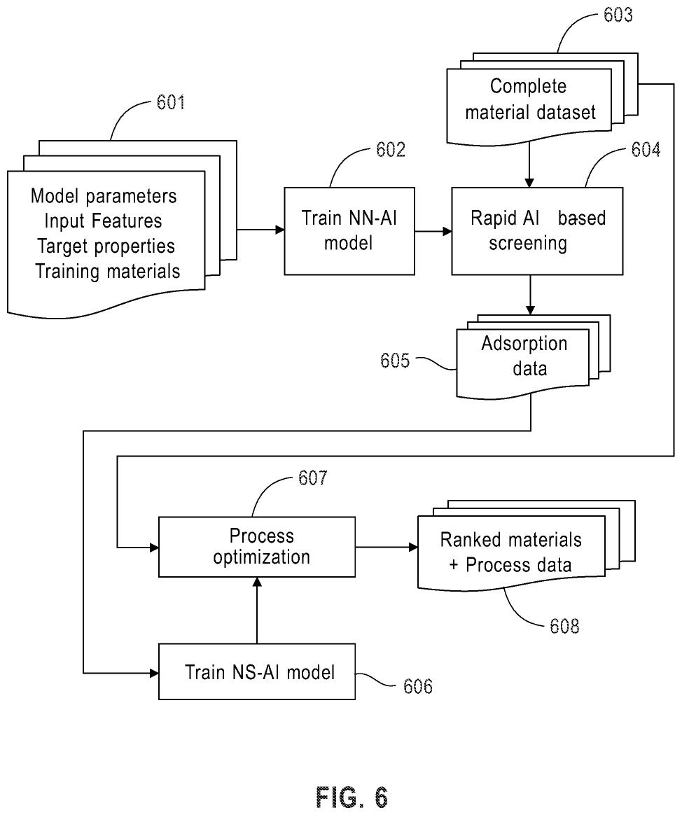

As shown in FIG. 6, in one embodiment, inputs 601 which may include model parameters, input features, target properties and training materials subset, are used to train a neural network-AI model similar to the process shown in FIG. 2. Then the complete material dataset 603 is used to run a rapid AI-based screening 604 using the trained neural network-AI model resulting in adsorption data 605. The adsorption data 605 is then used to train a neurosymbolic (NS)-AI model 606. Then a fraction of the complete material dataset is used to run the trained neurosymbolic (NS)-AI model 606 to perform process optimization 607. The ranked materials and process data are output at 608. This process combines symbolic and non-symbolic AI models for end-to-end high-throughput optimization of materials discovery and process engineering.

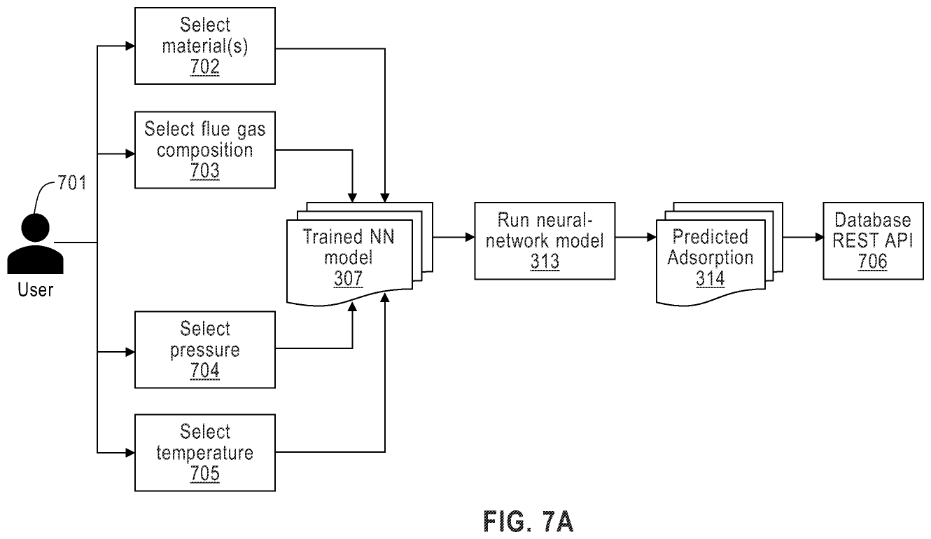

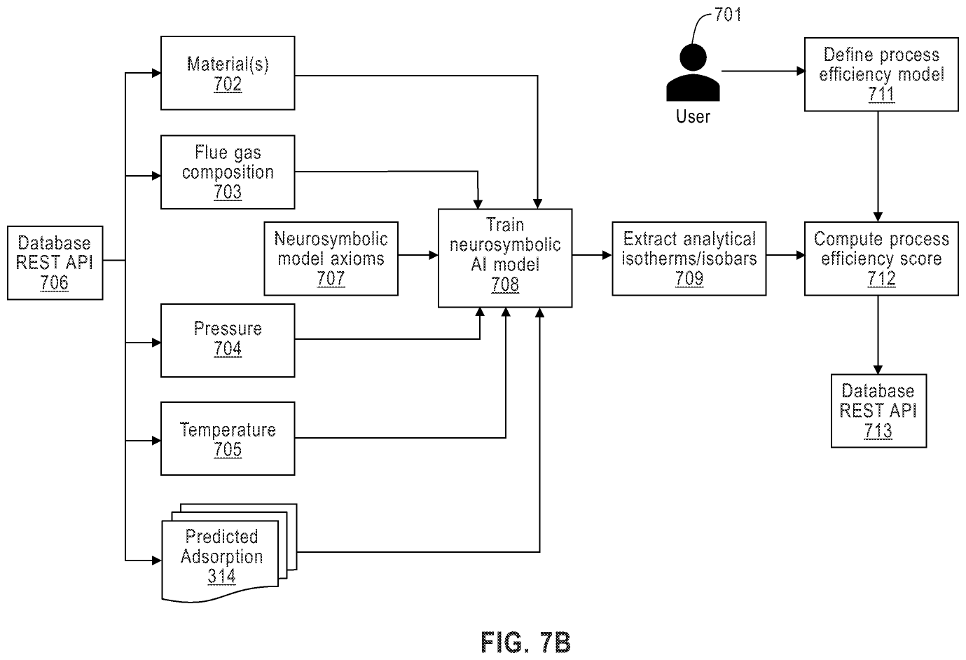

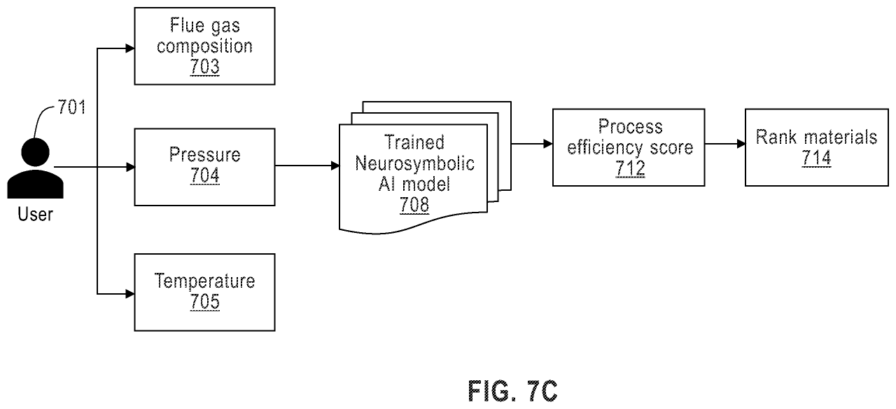

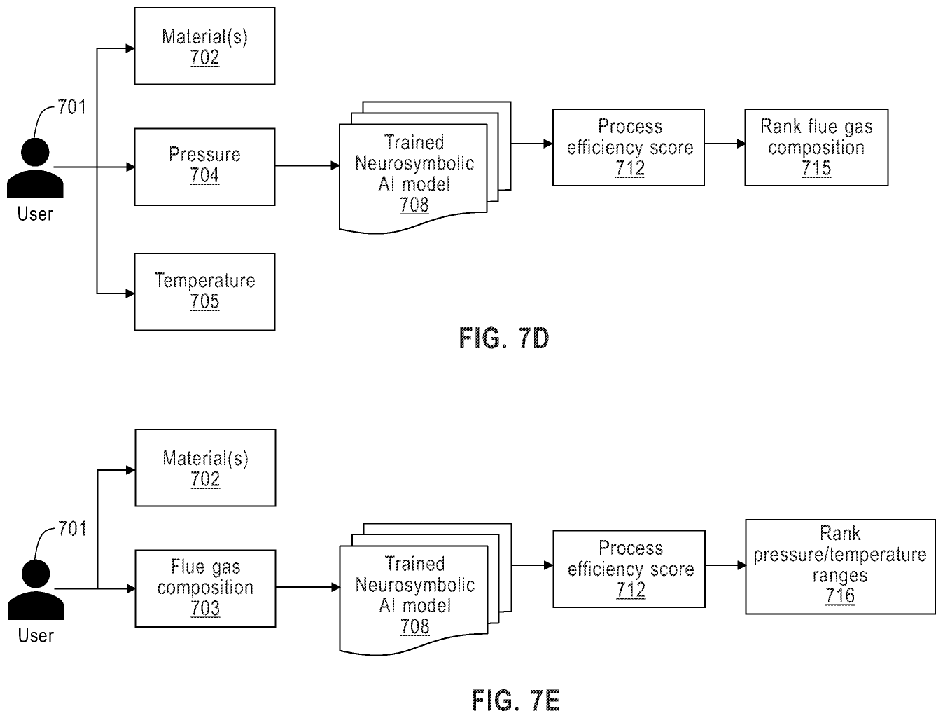

The corresponding framework for some embodiments of such an extension is outlined FIGS. 7A-7E. First, adsorption training data is generated as shown in FIG. 7A. The user 701 selects a variety of flue gas compositions 703, pressures 704 and temperatures 705 for different materials 702. The trained neural network 307 can then be deployed 313 to predict 314 the adsorption performance for each material 702, pressure 704, temperature 705 and flue gas composition 703. The estimated adsorption properties are added to the database 706.

Next, in some embodiments, neurosymbolic AI techniques can be applied to optimize process efficiency as shown in FIG. 7B. First, the training data is retrieved from the database 706. Next, neurosymbolic AI algorithms can be used to derive the analytical expressions which best fits the isotherm or isobar for each material as a defined function (f) of adsorption gas composition (c), pressure (P) and temperature (T). Note that f=f(c,P,T) is a closed form expression composed of a linear combination of discrete terms which are either added, subtracted, multiplied or divided with one another. Each individual term must have a simple mathematical form, such as an exponential, logarithmic, power law, sine, cosine etc. This set of potential mathematical forms which each term can take, is predefined as a set of axioms 707 that are used to train the neurosymbolic AI model 708.

In some embodiments, the fitted analytical expressions for the isobars/isotherms are then extracted 709 and evaluated 712 by computing a process efficiency score using a process efficiency model 711 defined by user 701. The process efficiency model 711 defines an engineering metric related to how efficient such a process would be. This evaluation can account for the economic cost of operating such a process as well as productivity measures related to the total number of captured carbon dioxide molecules. Finally, the process efficiency scores are written to the database 713.

FIGS. 7C-7E show how the process efficiency scores may be applied to optimize three distinct processing problems. In one embodiment, as shown in FIG. 7C, the flue gas composition 703, pressure 704 and temperature ranges 705 are fixed and used to train the neurosymbolic AI model 708. Process efficiency scores 712 of each material are calculated and the materials are ranked at 714 based on a combination of the adsorption data and the process efficiency data, to inform the operator 701 about which material sample they should use to maximize carbon capture. In one embodiment, as shown in FIG. 7D, the adsorbate material 702, pressure 704 and temperature ranges 705 are fixed and the system can calculate the process efficiency scores 712 of each flue gas composition at 715, to inform the operator about which flue gas composition they should use to maximize carbon capture. In one embodiment, the flue gas originating by the combustion process might contain a high amount of water vapor. The model might inform the operator that the carbon capture efficiency could be boosted by reducing the amount of water vapor in the flue gas stream. This information could lead the operator to perform a water-removal pre-treatment before triggering the adsorption process. In FIG. 7E, the adsorbate material 702 and flue gas composition 703 are fixed and the system can rank the process efficiency scores 712 of each prospective pressure/temperature range at 716, to inform the operator about which pressure/temperature range they should use to maximize carbon capture.

In some embodiments, the disclosed material screening methods and system accounts for the complete set of geometric, topological and chemical mechanisms which determine the results of molecular property simulations. In one embodiment, the neural network approach displays improved hierarchical screening performance compared to existing descriptors and operates as an initial screening layer capable of both regression and classification for subsequent screening steps. In one embodiment, the method eliminates the requirement for post-training simulations as the neural network will serve as a surrogate model for the simulation program. Therefore, the final screening process will be significantly faster than the current alternatives which still rely on performing physics-based simulations.

In some embodiments, the methods and systems disclosed provide hierarchical screening methods for scientific inference in materials research. The disclosed methods and systems are an improvement over the prior art by reducing the number of required physics-based simulations with both regression and classification capabilities. The method and systems disclosed allow for computationally screening large quantities of candidate materials with regards to pre-specified application figures of merits by combining AI and physics-based simulation techniques. The disclosed methods and systems have applications for climate study and materials discovery. The disclosed methods and system capture an automated, end-to-end molecular-level and process-level based screening framework. The methods and systems disclosed here can be generalized and applied to other material classes and separation processes.

In one embodiment, the methods and system are a cloud-based, AI enabled materials discovery screening system and method based on topological, geometrical and chemistry descriptors, capable to screening millions of potential nano-porous materials such as metal organic frameworks (MOFs) and Zeolites among others. The screening includes identifying crystalline, nano-porous materials with promising adsorption properties at varying pressures, temperatures, and gas compositions. The materials to be screened can be hypothetical or existing. The chemical processes to be screened can include isothermal or isobaric. The screening descriptors can include topological descriptors, geometrical descriptors, chemical descriptors, and physical descriptors.

In some embodiments, the screening method can be applied to identify candidate materials, such as Metal Organic Framework (MOFs), zeolites, zeolitic imidazolate frameworks, covalent organic frameworks or porous polymer networks. In some embodiments, the screening method can down select candidate materials for carbon capture and separation from CO₂ point source such as flue gas, natural gas and biogas upgrade. In some embodiments, the screening method integrates a surrogate artificial intelligence/machine learning model that replaces expensive and in-silico models. The screening methods and systems disclosed allow rapid and computationally efficient treatment of large quantities of candidate materials.

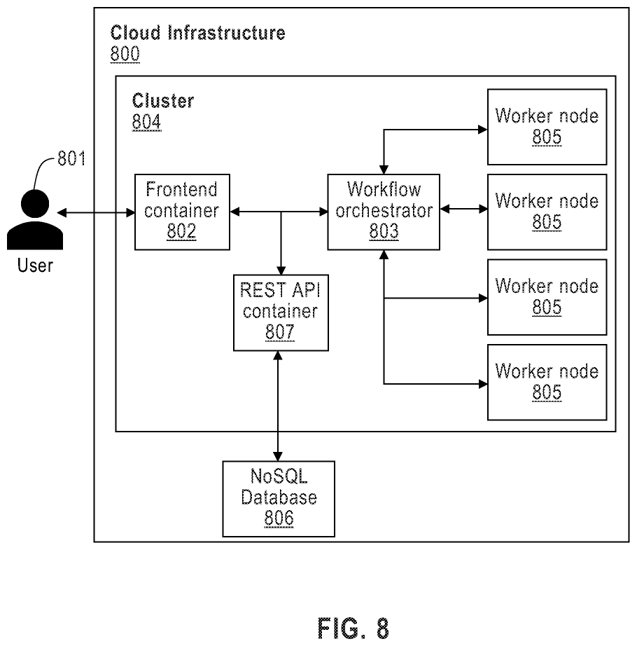

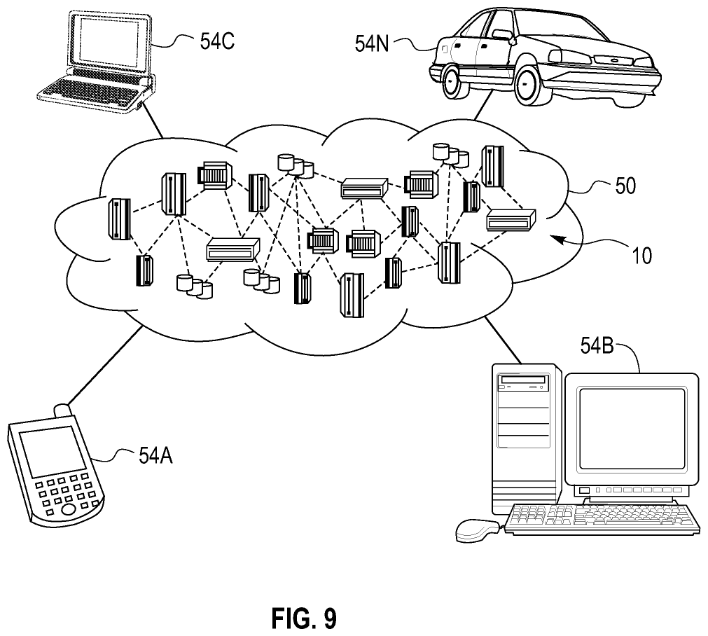

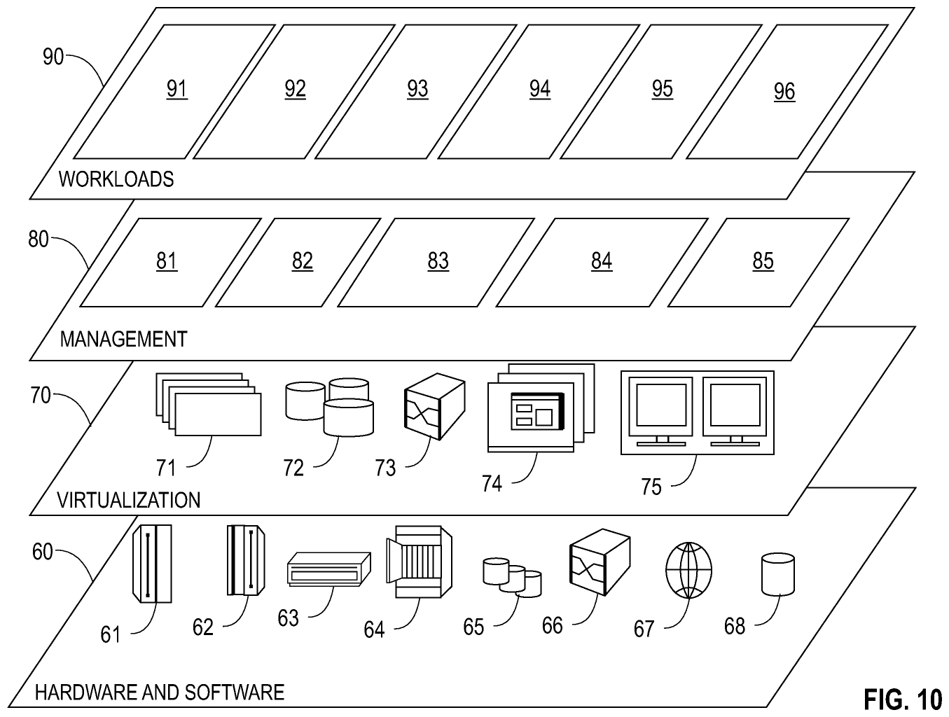

FIG. 8 is one embodiment of a cloud infrastructure 800 for implementing the methods and systems described in this disclosure. The user 801 interacts with the application through a front-end container 802 which uses the workflow orchestrator 803 to run multi-stage simulations on a cloud-computing cluster 804 with multiple worker nodes 805. The material properties resulting from these simulations are stored in a NoSQL Database 806 being hosted on the cloud 800. The REST API container 807 provides web endpoints to modify and query the database 806 from the front-end container 802. Further details of the cloud infrastructure 800 are described in connection with FIGS. 9 and 10 below.

It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load balancing between clouds).

A cloud computing environment is service oriented with a focus on statelessness, low coupling, modularity, and semantic interoperability. At the heart of cloud computing is an infrastructure that includes a network of interconnected nodes.

Referring now to FIG. 9, illustrative cloud computing environment 50 is depicted. As shown, cloud computing environment 50 includes one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 9 are intended to be illustrative only and that computing nodes 10 and cloud computing environment 50 can communicate with any type of computerized device over any type of network and/or network addressable connection (e.g., using a web browser).

Referring now to FIG. 10, a set of functional abstraction layers provided by cloud computing environment 50 (FIG. 9) is shown. It should be understood in advance that the components, layers, and functions shown in FIG. 10 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

In some embodiments of the cloud infrastructure of FIG. 8, the frontend container 802 may be implemented on layer 60 such as by a mainframe 61 or servers 62, 63 or 64. The REST API container 807 may be implemented on hardware and software layer 60, such as by the network application server software 67 and NoSQL database 806 may be implemented on database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75. In some embodiments of the cloud infrastructure of FIG. 8, the REST API container 807 and the NoSQL database 806 may be implemented virtualization layer 70.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA. In some embodiments of the cloud infrastructure of FIG. 8, the frontend container 802 and workflow orchestrator 803 may be implemented on management layer 80.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and materials screening layer 96. In some embodiments of the cloud infrastructure of FIG. 8, materials screening layer 96 includes the cloud-computing cluster 804 with multiple worker nodes 805 that run multi-stage simulations using the materials screening methods and systems described above.

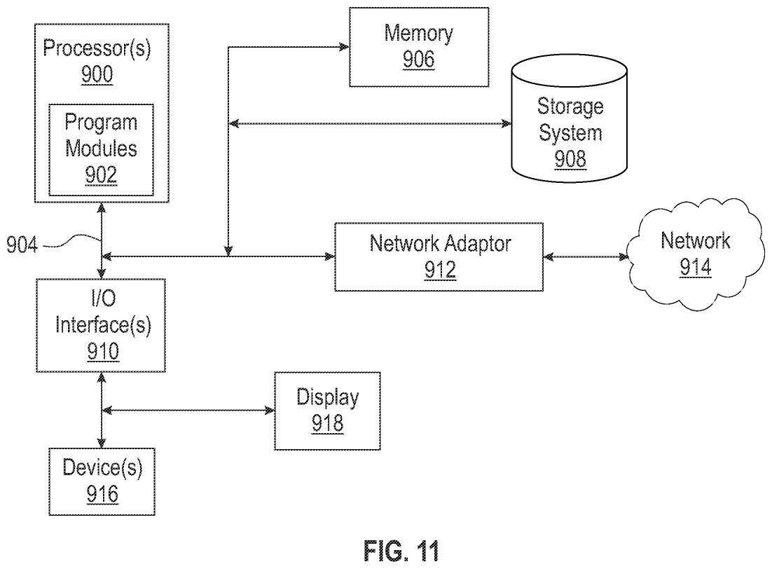

FIG. 11 illustrates a schematic of an example computer or processing system that may implement the methods for materials screening in some embodiments of the present disclosure. The computer system is only one example of a suitable processing system and is not intended to suggest any limitation as to the scope of use or functionality of embodiments of the methodology described herein. The processing system shown may be operational with numerous other general purpose or special purpose computing system environments or configurations. Examples of well-known computing systems, environments, and/or configurations that may be suitable for use with the processing system shown in FIG. 11 may include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

The computer system may be described in the general context of computer system executable instructions, such as program modules, being executed by a computer system. Generally, program modules may include routines, programs, objects, components, logic, data structures, and so on that perform particular tasks or implement particular abstract data types. The computer system may be practiced in distributed cloud computing environments where tasks are performed by remote processing devices that are linked through a communications network. In a distributed cloud computing environment, program modules may be located in both local and remote computer system storage media including memory storage devices.

The components of computer system may include, but are not limited to, one or more processors or processing units 900, a system memory 906, and a bus 904 that couples various system components including system memory 906 to processor 900. The processor 900 may include a program module 902 that performs the materials screening methods described herein. The module 902 may be programmed into the integrated circuits of the processor 900, or loaded from memory 906, storage device 908, or network 914 or combinations thereof.

Bus 904 may represent one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus.

Computer system may include a variety of computer system readable media. Such media may be any available media that is accessible by computer system, and it may include both volatile and non-volatile media, removable and non-removable media.

System memory 906 can include computer system readable media in the form of volatile memory, such as random access memory (RAM) and/or cache memory or others. Computer system may further include other removable/non-removable, volatile/non-volatile computer system storage media. By way of example only, storage system 108 can be provided for reading from and writing to a non-removable, non-volatile magnetic media (e.g., a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 104 by one or more data media interfaces.

Computer system may also communicate with one or more external devices 916 such as a keyboard, a pointing device, a display 918, etc.; one or more devices that enable a user to interact with computer system; and/or any devices (e.g., network card, modem, etc.) that enable computer system to communicate with one or more other computing devices. Such communication can occur via Input/Output (I/O) interfaces 910.

Still yet, computer system can communicate with one or more networks 914 such as a local area network (LAN), a general wide area network (WAN), and/or a public network (e.g., the Internet) via network adapter 912. As depicted, network adapter 912 communicates with the other components of computer system via bus 904. It should be understood that although not shown, other hardware and/or software components could be used in conjunction with computer system. Examples include, but are not limited to: microcode, device drivers, redundant processing units, external disk drive arrays, RAID systems, tape drives, and data archival storage systems, etc.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements, if any, in the claims below are intended to include any structure, material, or act for performing the function in combination with other claimed elements as specifically claimed. The description of the present invention has been presented for purposes of illustration and description but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

In addition, while preferred embodiments of the present invention have been described using specific terms, such description is for illustrative purposes only, and it is to be understood that changes and variations may be made without departing from the spirit or scope of the following claims.

What is claimed is:

1. A materials screening method comprising:
generating input features for each material of a subset of materials to be screened;
generating target properties for each material of the subset of materials;
inputting screening conditions, the input features, and the target properties into a material screening artificial intelligence model;
training the material screening artificial intelligence model based on the inputs;
inputting a dataset of materials to be screened into the trained material screening artificial intelligence model, the dataset of materials being larger than the subset of materials;
screening the dataset of materials on the trained material screening artificial intelligence model using the screening conditions; and
ranking the materials of the dataset based on predicted target properties obtained from the screening.

2. The method of claim 1, further comprising defining the subset of materials by a crystallographic information file for each material.

3. The method of claim 2, further comprising launching a virtual experiment using the screening conditions, retrieving a set of crystallographic information files representing a unit cell of each material and scanning each retrieved crystallographic information file for crystallographic disorder.

4. The method of claim 3, further comprising building a suitable stoichiometrically-balanced supercell with the appropriate size by replicating the unit cell as many times as necessary to avoid self-interactions.

5. The method of claim 1, wherein generating target properties for each material comprises determining adsorption metrics.

6. The method of claim 5, wherein determining adsorption metrics comprises assigning charges to each atom in the supercell, calculating electrostatic Ewald and van der Waals grids, launching a simulation using the screening conditions resulting in one of an adsorption isotherm, an adsorption isobar or an adsorption simulation for a single pressure and temperature value.

7. The method of claim 2, generating input features for each material comprises calculating topological and geometric metrics from the crystallographic information files.

8. The method of claim 1, further comprising training a neurosymbolic material screening model using predicted target properties, neurosymbolic axioms and the screening conditions, extracting analytical expressions of the target properties from the trained neurosymbolic material screening model, evaluating the extracted analytical expressions using a process efficiency model and calculating a process efficiency score.

9. A computer system for materials screening, comprising:
one or more computer processors;
one or more non-transitory computer-readable storage media;
program instructions, stored on the one or more non-transitory computer-readable storage media, which when implemented by the one or more processors, cause the computer system to perform the steps of:
generating input features for each material of a subset of materials to be screened;
generating target properties for each material of the subset of materials;
inputting screening conditions, the input features, and the target properties into a material screening artificial intelligence model;
training the material screening artificial intelligence model based on the inputs;
inputting a dataset of materials to be screened into the trained material screening artificial intelligence model, the dataset of materials being larger than the subset of materials;
screening the dataset of materials on the trained material screening artificial intelligence model using the screening conditions; and
ranking the materials of the dataset based on predicted target properties obtained from the screening.

10. The computer system of claim 9, further comprising defining the subset of materials by a crystallographic information file for each material.

11. The computer system of claim 10, further comprising launching a virtual experiment using the screening conditions, retrieving a set of crystallographic information files representing a unit cell of each material and scanning each retrieved crystallographic information file for crystallographic disorder.

12. The computer system of claim 11, further comprising building a suitable stoichiometrically-balanced supercell with the appropriate size by replicating the unit cell as many times as necessary to avoid self-interactions.

13. The computer system of claim 9, wherein generating target properties for each material comprises determining adsorption metrics.

14. The computer system of claim 13, wherein determining adsorption metrics comprises assigning charges to each atom in the supercell, calculating electrostatic Ewald and van der Waals grids, launching a simulation using the screening conditions resulting in one of an adsorption isotherm, an adsorption isobar or an adsorption simulation for a single pressure and temperature value.

15. The computer system of claim 10, generating input features for each material comprises calculating topological and geometric metrics from the crystallographic information files.

16. The computer system of claim 9, further comprising training a neurosymbolic material screening model using predicted target properties, neurosymbolic axioms and the screening conditions, extracting analytical expressions of the target properties from the trained neurosymbolic material screening model, evaluating the extracted analytical expressions using a process efficiency model and calculating a process efficiency score.

17. A computer program product comprising:
program instructions on a computer-readable storage medium, where execution of the program instructions using a computer causes the computer to perform a method for materials screening, comprising:
generating input features for each material of a subset of materials to be screened;
generating target properties for each material of the subset of materials;
inputting screening conditions, the input features, and the target properties into a material screening artificial intelligence model;
training the material screening artificial intelligence model based on the inputs;
inputting a dataset of materials to be screened into the trained material screening artificial intelligence model, the dataset of materials being larger than the subset of materials;
screening the dataset of materials on the trained material screening artificial intelligence model using the screening conditions; and
ranking the materials of the dataset based on predicted target properties obtained from the screening.

18. The computer program product of claim 17, further comprising defining the subset of materials by a crystallographic information file for each material and wherein generating target properties for each material comprises determining adsorption metrics by assigning charges to each atom in a supercell, calculating electrostatic Ewald and van der Waals grids, launching a simulation using the screening conditions resulting in one of an adsorption isotherm, an adsorption isobar or an adsorption simulation for a single pressure and temperature value.

19. The computer program product of claim 18, further comprising launching a virtual experiment using the screening conditions, retrieving a set of crystallographic information files representing a unit cell of each material and scanning each retrieved crystallographic information file for crystallographic disorder, and building a suitable stoichiometrically-balanced supercell with the appropriate size by replicating the unit cell as many times as necessary to avoid self-interactions.

20. The computer program product of claim 17, further comprising training a neurosymbolic material screening model using predicted target properties, neurosymbolic axioms and the screening conditions, extracting analytical expressions of the target properties from the trained neurosymbolic material screening model, evaluating the extracted analytical expressions using a process efficiency model and calculating a process efficiency score.
